(19) United States
(12) Patent Application Publication
(10) Pub. No.: US 2019/0275097 A1
(43) Pub. Date: Sep. 12, 2019

(54) Lycopene from Momordica Cochinchinnensis Spreng., methods and formulations

(71) Applicant: Le Thuy Vuong, Elk Grove, CA (US)

(72) Inventor: Le Thuy Vuong, Elk Grove, CA (US)

(21) Appl. No.: 15/917,055

(22) Filed: Mar. 9, 2018

(51) Int. Cl. A61K 36/42; A61K 31/01; A61K 31/355

(52) U.S. Cl. CPC A61K 36/42; A61K 31/01; A61K 2236/37; A61K 2236/15; A61K 2236/17; A61K 31/355

(57) ABSTRACT

Momordica Cochinchinensis Spreng. (Gac, spiky groud, redmelon) contains high concentration of lycopene, a natural pigment that has many applications in pharmaceuticals, food supplements and colorants. This invention describes methods to extract lycopene from Gac. The methods do not involve the use of harmful solvents, therefore safe for consumption, and produce no toxic residue, nor toxic waste. This invention provides solutions to populations whose intake of lycopene is low, or having problems with high acid foods. This invention provides formulations high in lycopene, other antioxidants, and low in acid, and safe for consumers. The formulations described herein can be applied in beverage, food, supplements, pharmaceuticals, cosmeticals and nutraceuticals.

### 1. PARENT CASE TEXT AND RELATED APPLICATION

This application is a continuation-in-part of U.S. Pat. No. 6,770,585 (application Ser. No. 10/211,814 filed Aug. 5, 2002) and of U.S. patent application Ser. No. 10/163,280 filed on Jun. 06, 2002, now abandoned, which claims the benefit of U.S. Provisional Patent Application Ser. No. 61/688,596 filed on May 18, 2012, and non-provisional patent application Ser. No. 13/897,279 filed May 17, 2003, all of which are incorporated herein by reference.

### Related U.S. Patent Documents

| Application Number | Filing Date | Patent Number | Issue Date |
|---|---|---|---|
| 10/211,814 | Aug. 5, 2002 | 6770585 | 2004 |
| 10/163,280 | Jun. 6, 2002 | | |

### 2. FIELD OF INVENTION

The present invention relates to beverage, food, fruit drinks, fruit concentrates, and nutritional drinks. More particularly, the invention relates to methods and formulation containing lycopene from fruit. Accordingly, the invention involves the fields of botany, nutritional and health sciences, and medicine.

### 3. OBJECTS AND ADVANTAGES

The objects of this invention describes methods and formulations containing, stable and bioavailable lycopene from Momordica cochinchinensis Spreng (spiny melon). The formulations contain no chemical solvents, and thus safe for human and animal consumption. The formulations can be applied to beverage, food, pharmaceuticals, cosmetics industries as well as providing a suitable, effective food additive and colorant.

Spiny melon is indigenous to Southeast Asia. The plant can be cultivated either from seeds or root tubers. Seed pulp of spiny melon contains a significant amount of carotenoids. The dry pulp which is a residue of oil extraction is usually discarded. In one of the embodiments of this invention, the seed pulp after dried and oil is removed, is further processed to produce a composition high in lycopene. There is no organic solvent used in this process and thus the product is safe for human and animal consumption. Another advantage of this invention is that the process used to obtain lycopene is simple and can be applied in rural areas.

Lycopene is among hydrocarbon carotenoids with antioxidative activities. According to epidemiological and animal studies, these carotenoids play an important role in the prevention of cancer, cataracts, and degenerative diseases such as heart disease. It would be a significant advancement in the art to obtain lycopene from Momordica cochinchinensis fruits in regions where this plant is widely grown.

### 4. BRIEF SUMMARY OF THE INVENTION

This invention provides methods to extract lycopene from the fruit Momordica cochinchinnesis Spreng. or redmelon or gac fruit. In particular, mechanical and non-toxic solvent methods to extract lycopene and to produce a bioavailable and stable lycopene. In some aspects, the products can be either in oil or dry powder form. The formulations and associated method are characterized by the present of other antioxidant carotenoids, such as alpha-carotene, beta-carotene, vitamin E which make the lycopene stable. The invention also described formulations containing high lycopene however low in acid, and thus more bio-accessible, or bio-available. Products derived from this invention serve as a stable and bioavailable source of lycopene to be incorporate in diet or beverage or as nutritional supplement for human and animal consumption, for the pharmaceuticals and cosmetics and ingredients industries as a suitable, effective food additive and colorant.

### 5. BACKGROUND AND PRIOR ART

Lycopene is the major carotenoid in human plasma, tissue and diet and exhibits the highest antioxidant activity and singlet oxygen quenching ability of all dietary carotenoids (Mascia et ai.). According to epidemiological and animal studies, Lycopene plays an important role in the prevention of cancer, cataracts, and degenerative diseases such as heart disease (Bendich, Giovannucci et ai., Levy et ai., Narisawa et ai.). Humans can not make lycopene but obtain lycopene from diet, and the richest source of lycopene in western diet are tomatoes and tomato products (Gartner et ai., Khachik, Sadler et ai., Tan). Typically lycopene amounts to 85-90% by weight of the carotenoids in tomatoes. Concentrations of lycopene in common tomatoes ranges from 30 to 100 ppm, on the average at 50 ppm, fresh weight basis Pure lycopene extracted from tomatoes frequently is re-suspended in vegetable oil. Tomatoes products contain high acid level, and thus are not palatable for some populations. In Momordica cochinchinensis (redmelon or gac fruit), concentration of lycopene ranges from 250 ppm to 700 ppm and on the average 450 ppm.

Zelkha et al. describes a process in which crushed, heated tomatoes are fractionated into serum and pulp containing; the lycopene is extracted from the pulp using a solvent. The solvents listed by patentees as satisfactory are hexane, ethyl acetate, and dichloromethane; however, neither dichloromethane nor hexane are environmentally friendly.

U.S. Pat. No. 5,871,574 to Kawaragi et al. uses an enzymatic technique to macerate tomato pulp to facilitate easier downstream processing. Their downstream processing entails centrifuging to recover the liquid portion and then passing this liquid through a microfilter and collecting the retentate. This enables an appropriate microfractionation to optimize pigment size. The crude pigment may be further purified by agitation in 75% organic solvent such as methanol, ethanol, propanol, or acetone, and then recovering the sediment. The sediment is then dried to yield a tomato pigment.

U.S. Pat. No. 5,897,866 to Bombardelli et al. describes a process to obtain lycopene wherein partially dehydrated whole fruits of Lycopersicum esculentum (tomato) are extracted with a solvent in the presence of phospholipids as surfactant and stabilizing agents and the extracts are concentrated or fractionated to an oil. The preferred solvents are n-hexane and methylene chloride, and the preferred phospholipid is soy lecithin.

A water-based method has been used to extract lycopene from tomato pulp. Extracting lycopene from tomatoes seeds and peel using SFE have been published. This technique is safe and ecological friendly, however, costly, and so far, has not been carried out in commercial quantity. PCT Application No. WO 01/38443 A1 to Bortlik et al. describe a process for extraction of lycopene from tomatoes and tomato pomace using boiling ethanol.

U.S. Pat. No. 6,818,239 to Kagan et al. describes a process for the extraction of carotenoids from a carotenoid source such as a biomass, which comprises treating the carotenoid source at an elevated temperature with a solvent mixture comprising water, a hydrophobic carotenoid solvent such as vegetable oil and a water soluble co-solvent such as ethanol so as to extract the carotenoid source into the hydrophobic solvent.

U.S. Pat. No. 5,714,658 to Heidlas et al. describes extraction of carotenes from natural sources, by contacting the starting material with a solvent mixture composed of at least one acetic ester of C.sub.1-C.sub.4 alcohols and 1 to 25% by weight of an oil of biological origin at a temperature of at least 30 degree C.

U.S. Pat. No. 5,789,647 to Heidlas et al. describes extraction of carotenoid dyes from pre-dried natural starting materials using compressed gases such as propane or butane and optionally an organic entraining agent such as acetone, ethyl acetate, or ethanol.

U.S. Pat. No. 5,245,095 to Graves et al. describes a process of extraction of carotenoids from carrots wherein a carotenoid-precipitation agent including calcium chloride, calcium hydroxide, calcium lactate, or calcium gluconate is added to a liquid fraction obtained from a carotenoid-containing natural source to form a carotenoid-enriched solid precipitate.

U.S. Pat. No. 5,830,738 to Thomas et al. describes a process for extracting carotenoids from pigmented plant material, which includes contacting shredded plant material with an enzyme.

Processes to extract carotenoids such as lycopene using supercritical CO.sub.2 have been described. See Ollanketo et al., European Food Research Technologies 212: 561-565, 2001, Rozzi et al., Journal of Agriculture and Food Chemistry 50:2638-2643, 2002, and U.S. Patent Applications 20030180435 and 20040131733.

Methods to produce water soluble carotenoids and lycopene have been described. Hills et al. in U.S. Pat. No. 4,851,339 filed in July 25, 1989 described methods to extract carotenoids from agae to produce a water soluble product. Koguchi et al. in U.S. Pat. No. 6,261,622 filed on Jul. 17, 2001 and Furuguchi in patent number JPH1160980 (A) filed in Mar. 5, 1999 described methods to extract carotenoid pigment and dispersed in soybean fiber.

None of the above patents described methods applicable to Momordica cochinchinensis. Formulations using “gac” fruit were published—Smidt et al. in U.S. Pat. No. 8,067,045 filed on Nov. 29, 2011 described nutritional formulations of “gac” extract and other plant extract to enhance absorption of antioxidants. Mower et al. described the use of Momordica cochinchinensis puree as a sweetener, to mask the bitterness of other phytonutrients in the composition. In U.S. Pat. No. 5,942,233 by Chang, Teh Shan filed on Aug. 24, 1999 titled “Herbal composition for stimulating blood circulation”. This invention described the use of Momordica cochinchinensis as one of the components in a paste that is useful for re-establishing of vital energy, invigorating of blood circulation. In U.S. Pat. No. 8,067,045 filed in Nov. 2011, Smidt et al. described the use of “gac” fruit extract as a component in a nutritional formula improve absorption of antioxidants.

Other fruits also provide lycopene, however in small amount, such as watermelon, persimmon, papaya, guava, pitanga, rosehip. Efficient methods to extract lycopene from those sources involve the use of chemical solvents, the products are not deemed food grade and should not be used in food or supplements for human or animal consumption. Further, the method can not be applied in regions of the world where chemicals or high technological instruments are not available.

Bioavailability is the amount of bioactive components in diet that is available to the target tissues. Bioavailability of lycopene depends on many factors: concentration in the food, the food matrix (oil based is more bioavailable, fibrous or water based is not as bioavailable), type of food, the presence of other nutrients (vitamin E, vitamin C have been shown to be enhancing); type of delivery or packaging since lycopene can be destroyed or reduced its bioactivity by exposed to air, high heat, cooking technique (high heat isomerizes lycopene, reducing its bioavailability); The present of fatty acids is important in the absorption and deliver of lycopene to target tissue (Erdman), and medium chain fatty acids and unsaturated can improve absorption (Conlon L E, King R D, Moran N E, Erdman J W Jr) Lycopene in fruits has been shown to be more bioavailable than lycopene in vegetables (Suskia de Pee).

Another factor that involves in the taste of the dietary source of lycopene is acid level. Tomatoes are generally considered a high acid food item with a pH below 4.6. Unfortunately, a lot of misinformation has been printed in the popular press about “low acid” tomatoes referring to those with a sweet, non-tart taste. These tomatoes are often white, yellow, or pink in color, low in lycopene but are not low in acid content. Red colored tomatoes, although containing high level of lycopene, high acid level in tomatoes makes this source less attractive for applications in beverage and for target populations who can not tolerate high acid food. The distinctive taste of tomato also make this source of lycopene unpalatable in fruit juice or sweet drink.

Another problem of this source of lycopene is that tomatoes contain very little oil, lycopene extract from tomatoes needs to be re-suspended in oil, and can only be applicable to limited markets, such as food supplements.

In regions where tomatoes are not commonly grown, and canned foods are not widely available, people of low economic groups have limited access to food rich in lycopene. Furthermore, high acid level in tomatoes makes this source not palatable in many applications. Low fatty acids in tomatoes also make this source of lycopene unstable, and not bioavailable. In populations with low source of fat consumption, this source of lycopene is not practical.

Momordica cochinchinensis Spreng (spiny melon, gac, redmelon) is indigenous to Southeast Asia and Australia. Descriptions of the plant can be found in several publications (Bailey, Heiser, Herklots and Perry). Medicinal use of Momordica cochinchinensis seeds and seed pulp, and other parts of the plant was described by Nguyen, and by Vuong (1998).

Concentrations of total carotenoids in Momordica cochinchinensis (17000 to 75000 mcg/100 g wet weight) can be found in a publication by West & Poortvliet. Lycopene is the main carotenoid in the fruit (8000 ppm, or 80% to 92% of total carotenoids and beta-carotene is another major carotenoid (4-6% of total carotenoids). A publication by Vuong described the plant and discussed its use in improving lycopene status of children in Vietnam (2000). Another publication by Vuong et al. described a supplementation trial of rice mixed with Momordica cochinchinensis pulp to improve plasma retinol and beta-carotene of preschoolers in Vietnam (Vuong et ai., 2002).

Method to extract oil from Momordica cochinchinensis spreng using cold press has been provided in U.S. Pat. No. 6,770,585. The oil contains beta-carotene 4100 ppm and approximately 550 ppm lycopene. Another method which uses chemical solvent to extract carotenoids from “gac” was published in U.S. Pat. No. 7,572,468. As described in this method, the extraction solvent comprises a blend of ethyl lactate and ethanol. This method of extraction of carotenoids from Momordica cochinchinensis used chemical solvents and therefore products are not deemed to be safe for consumption.

EU Patent CN101611878 (A) by Dayan, et al. filed on Dec. 30, 2009 described method for preparing carotenoids in “gac” fruit using alcohol. Although the invention appeared to use no toxic chemicals, the method described the use of alcohol, or anhydrous sodium sulfate, or ethyl acetate. This method also described the “gac” fruit as a “wood turtle fruit”, which did not correctly describe the fruit Momordica cochinchinensis Spreng.

Other foreign patents on the use of Momordica cochinchinensis described the use of components inside the seed of the fruit in the treatment of skin disorders (Patent CN101697993(A); Patent CN101219173 (A); CN101219173 (B) filed Jul. 16, 2008 by HAIJIANG XIA [CN] described a mixture of Chinese ephedra, radix sileris, cortex lycii radicis, dictamni cortex and Momordica cochinchinensis seed to treat psoriasis; Patent JP2010001264 (A) filed in Jan. 7, 2010 by Takano Kenichi, Kurachi Michio and Noguchi Yuki described the use of Momordica cochinchinensis seed among other plants as “nerve stretch-inhibiting agent”; Patent JP2009040691 (A) filed on Feb. 26, 2009 by Takagaki et al. described solutions using Momordica cochinchinensis seed coat and extract of seed coat with solvent as skin lighting ingredient; Patent JPH09328434 (A) filed Dec. 22, 1997 by Kubo et al. described the use of dried root of Momordica cochinchinensis (Lour.) Spreng.) extracted with alcohol or acetone as an anti-itch agent.).

In view of the foregoing, methods and formulations which can deliver safe (no chemical solvents), stable and bio-available lycopene in a low acid formulations, that can impart positive health benefits to humans and animals will be discussed.

### 6. DETAILED DESCRIPTION OF INVENTION

This invention describes methods to extract lycopene from Momordica cochinchinensis Spreng. (Gac, spiky groud, redmelon). The methods do not involve the use of harmful solvents, therefore safe for consumption, and produce no toxic residue, nor toxic waste. This invention provides solutions to populations whose intake of lycopene is low, or having problems with high acid foods. The invention described compositions high in lycopene, other antioxidants, and low in acid, and safe for consumers. The compositions described herein can be applied in beverage, food, supplements, pharmaceuticals, cosmeticals and nutraceuticals.

Before particular embodiments of the present invention are disclosed and described, it is to be understood that this invention is not limited to the particular process and materials disclosed herin as such may way to some degree. It is also to be understood that the terminology used herin is used for the purpose of describing particular embodiments only and is not intended to be limiting.

### Definition of Terms

In describing and claiming the present invention, the following terminology will be used.

The term “Gac”, “redmelon™”, “gac fruit” refer to the plant Momordica cochinchinensis Spreng, or of plants significantly related thereto, grown anywhere in the world including blends, mixtures, and combinations of such strains and relatives.

The singular forms “a,” “an,” and “the” include plural referents unless the context clearly dictates otherwise. As used herein, “formulation” and “composition” may be used interchangeably herein, and refer to a combination of two or more elements, or substances. In some embodiments a composition may include an active agent and a carrier.

As used herein, the terms “bioavailable” or “bioavailability” when used in connection with antioxidants refer to the antioxidant portion of a formulation which is available to be absorbed or taken up by the subject.

As used herein the term “stability” when used in connection with carotenoids refer to the portion of the carotenoids in the formulations that is not destroyed or oxidized or loss and available for the uptake by a subject.

### 7. BRIEF DESCRIPTION OF DRAWINGS

Ranges of lycopene concentrations in a fresh Gac fruit (mg/100 g of wet weight) are listed in TABLE 1.

### 8. DETAILED DESCRIPTION OF THE INVENTION

The novelty of the invention is in the use of the seed pulp of Momordica cochinchinensis to obtain lycopene. Lycopene obtained this way is stable since it is contained in a lipid matrix high in alpha-tocopherol and other antioxidants, and bioavailable. Further, the methods described do not involve chemical solvents, and therefore the products are safe for human and animal consumption, without any toxic residue, nor toxic waste.

Momordica cochinchinensis Spreng (spiny melon) is botanically classified as Family Cucurbitaceae, Genus Momordica, Species cochinchinensis. This plant is indigenous to Southeast Asia and Australia. In one currently preferred embodiment of the invention, a quantity of Momordica cochinchinensis fruits is obtained. Rind, Pulp and seed pulp (Aril) are removed from seeds, and dried in an electric powered drying box under 60° C. The dried Momordica cochinchinensis pulp preferably has a moisture content in the range of 10-15% by weight. Aril is separated from rind and pulp. In one currently preferred embodiment of the invention, dried aril is heated slightly before pressing for oil using an oil expeller. Oil extract of Momordica cochinchinensis aril contains from 3000 to 6500 ppm of carotenoids by weight, of which 45-75% are beta-carotene. The dried Momordica cochinchinensis pulp, after oil is removed, preferably has a moisture content in the range from 0.1% to 10% by weight and about 85% of total carotenoids is lycopene. The dried aril, rind and pulp may be further processed by drying, pulverizing, and purifying, The methods of drying consist of but are not limited to freeze drying (lyophilizing), drum drying, tray drying, sun drying, and spray drying. Further extraction of lycopene from the oil free aril, rind and pulp can be done using supercritical fluid extraction. Purification of lycopene after extraction can be done by recrystalization. The preferable process to totally utilize the nutrients in the pulp including fiber and maximize bioavailability of carotenoids (lycopene, betacarotene, lutein) is to reduce particle size by any commercial grinding or pulverizing equipment. The powder then can be packaged in air-tight, light proof containers for storage, transport and/or further processing. This lycopene rich from Momordica cochinchinensis can be used in a variety of applications, including, but not limited to cosmetics, nutritional products, dietary supplements, as a product itself such as snack food, or flavoring, or in animal feedstuffs.

In one embodiment of the invention, a quantity of Momordica cochinchinensis seed pulp is obtained either with or with out seeds. Pulp can obtained from fresh or frozen fruit, frozen whole fruit or frozen pulp. Seeds can be removed either manually or mechanically, and dried by air or in an electric powered drying box under 60° C. The dried Momordica cochinchinensis (also known as gac) pulp (aril) preferably has a moisture content in the range of 10-15% by weight. In another embodiment of the invention, dried aril is heated slightly before pressing for oil by an oil expeller. Oil extract of Momordica cochinchinensis pulp contains from 3000 to 6500 ppm of carotenoids by weight, of which 45-75% are beta-carotene. Dried pulp after pressed contains about 4500 ppm of lycopene (about 85% of total carotenoids), and approximately 2%-5% of oil.

Dried-deoiled seed pulp contains 17-20% water is dried further in low heat, to reduce water, and pulverized to produce lycopene powder. This method can be applied in rural setting without use of food processing equipments. In one embodiment, dried-deoiled seed pulp is mix with coconut oil, which is wildly available, to produce a formula rich in vitamin E, and lycopene. This formulation provides stable and bioavailable lycopene.

Further extraction and purification of lycopene using supercritical fluid extraction method with CO2 can be applied.

The fruits selected should be ripen, meaning when the exocarp is 90% orange, and should not show any evidence of infested or rotten. Fruits were washed and opened. Seed membrane was removed by hand. In another embodiment, seed membrane was removed and dried in box dryer before seeds were removed. If not processed instantly, seed membrane should be stored in sealed, dark container at subzero freezer. Suboptimal transportation or storage conditions will cause isomerization and degradation of lycopene. Seed membrane obtained from this method show a high concentration of lycopene (408 ppm), compared to tomatoes (25 ppm).

Concentrations of lycopene in different parts of a fresh Momordica cochinchinensis Spreng. (Gac fruit) are shown in the following table:

| Parts of a whole | Lycopene concentrations |
|---|---|
| fruit | (mg/100 g) |
| Aril (seed pulp) | 154-305 |
| Rind (skin) | 38.4-81.6 |
| Pulp | 14.4-49.6 |
| Total | 206.8-436.2 |

TABLE 1. Ranges of lycopene concentrations in a fresh Gac fruit (mg/100 g of wet weight)

Values presented are representative of quantitative analyses for this invention, but not inclusive or absolute, since nutrient contents of fruits may vary depending on ripeness, growing regions or climate and post-harvest handling and processing.

It will be readily apparent to those skilled in the art that various changes and modifications of an obvious nature may be made without departing from the spirit of the invention, and all such changes and modification s are considered to fall within the scope of the invention as defined by the appended claims. Such changes and modifications would include, but not be limited to, the incipient ingredients added to affect the capsule, tablet, lotion, food or bar manufacturing process as well as vitamins, herbs, flavorings and carriers. Other such changes or modifications would include the use of other additives or botanical products containing the combinations of the preferred embodiments disclosed above. Many additional modification s and variations of the embodiments described herein may be made without departing from the scope, as is apparent to those skilled in the art. The specific embodiments described herein are offered by way of example only.

In one embodiment, seed membrane were lyophilized until water content was between 0.01-0.1%. The lyophilized (freeze dried) membrane has mildly sweet taste and can be easily mixed in beverage or food stuff.

The good absorption and transport of carotenoids after gac pulp consumption are probably due to its soft texture and to these high levels of fatty acids. Even when Gac pulp is consumed alone, lycopene and beta-carotene are highly bioavailable, as demonstrated by a significant increase in the human circulation after a 30-day feeding trial. Bioaaccessibility studies using in-vitro digestion/caco cell techniques also showed a high bioavailability for these carotenoids. This is in sharp contrast to other carotenoid rich fruits, which show a relatively low carotenoid bioavailability due to the lack of uptake enhancing lipids.

### EXAMPLES

The following examples are given to illustrate various embodiments which have been made or may be made in accordance with the present invention. These examples are given by way of examples only, and it is to be understood that the following examples are not comprehensive or exhaustive of the many types of embodiments of the present invention which can be prepared in accordance with the present invention.

Example I. A rice preparation rich in lycopene:

| Ingredients | Amount | |
|---|---|---|
| Gac (redmelon) powder | ½ | cups |
| Sushi rice (glutinous rice) | 2 | cups |
| Gac (redmelon) oil extract | 2 | Tablespoon |
| Sugar | 1 | tablespoon |
| Roasted sesame seeds | 1 | tablespoon |

Mix powder and oil together. Mix rice and 2 cups water in a rice cooker, and cook per instruction. When rice is cooked, drizzle oil extract, sugar and sesame seeds, mix well. Can substitute sugar and sesame seeds with dried sweetened grated coconut (½ cups).

Example 2. A composition containing Momordica cochinchinensis oil (Mc oil) and dried pulp (Mc pulp), rich in b-carotene and lycopene was prepared for animal feeds.

Add to a Corn-soybean meal based pig diet (15.9% Crude Protein; 0.79% lysine; 3,328 kcal ME/kg) the following ingredients :

| | | |
|---|---|---|
| Mc pulp | 5 | g/kg |
| Mc oil | 10 | ml/kg |

It is to be understood that the above-described embodiments are only illustrative of the application of the principles of the present invention. Numerous modifications and alternative formulations can be devised by those skilled in the art without departing from the spirit and scope of the present invention. Thus, while the present invention has been fully described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

### REFERENCES CITED

U.S. Patent Documents

| | | |
|---|---|---|
| 3172770 | March 1965 | Miller |
| 5245095 | September 1993 | Graves et al. |
| 5274073 | December 1993 | Gruber et al. |
| 5462695 | October 1995 | Tsai et al. |
| 5686489 | November 1997 | Yu et al. |
| 5714658 | February 1998 | Heidlas et al. |
| 5773075 | June 1998 | Todd |
| 5789647 | August 1998 | Heidlas et al. |
| 5830738 | November 1998 | Thomas et al. |
| 5837311 | November 1998 | Zelkha et al. |
| 5871574 | February 1999 | Kawaragi et al. |
| 5897866 | April 1999 | Bombardelli et al. |
| 6818239 | November 2004 | Kagan etal. |
| 2003/0052424 | March 2003 | Turner et al. |
| 2003/0061761 | April 2003 | Nanninga et al. |
| 2003/0180435 | September 2003 | Shi |
| 2004/0115260 | June 2004 | Schmid et al. |
| 2004/0131733 | July 2004 | Rey et al. |
| 2004/0208983 | October 2004 | Hill et al. |
| 2008/ | August 2008 | Ishida et al. |
| 20070212433 | Sept, 2007 | Smidt et al. |
| 8,017,147 | Sept. 13, 2011 | Mazed, et at |

Foreign Patent Documents

| | | |
|---|---|---|
| JP 53118597 | Oct., 1978 | |
| CN101611878 | Dec. 30, 2009 (A) | Dayan, et al. |
| JPH1160980 (A) | Mar. 5, 1999 | Furuguchi, et al. |

U.S. Patents (reference list only)

| | | |
|---|---|---|
| 7,781,572 | Bartlett, et al. | Aug. 24, 2010 |
| 8,420,147 | Abelyan, et al. | Apr. 16, 2013 |
| 8,414,950 | Abelyan, et al. | Apr. 9, 2013 |
| 8,420,146 | Abelyan, et al. | Apr. 16, 2013 |
| 8,414,952 | Abelyan, et al. | Apr. 9, 2013 |
| 8,414,951 | Abelyan, et al. | Apr. 9, 2013 |
| 8,414,949 | Abelyan, et al. | Apr. 9, 2013 |
| 8,414,948 | Abelyan, et al. | Apr. 9, 2013 |
| 8,337,858 | Scoglio, et al. | Dec. 25, 2012 |
| 8,298,548 | Avila, et al. | Oct. 30, 2012 |
| 8,277,849 | Dillon, et al. | Oct. 2, 2012 |
| 8,246,961 | Arakawa, et al. | Aug. 21, 2012 |
| 8,075,929 | Shrikhande, et al. | Dec. 13, 2011 |
| 8,017,162 | Shimoda, et al. | Sep. 13, 2011 |
| 7,993,654 | Woeller, et al. | Aug. 9, 2011 |
| 7,867,545 | Friedman | Jan. 11, 2011 |
| 7,820,177 | Kruse et. al. | Oct. 26, 2010 |
| 7,767,235 | Shrikhande, et al. | Aug. 3, 2010 |
| 7,651,707 | Kappagoda | Jan. 26, 2010 |
| 7,588,781 | Bortlik | Sep. 15, 2009 |
| 7,348,034 | Murray, et al. | Mar. 25, 2008 |
| 7,186,422 | Jensen, et al. | Mar. 6, 2007 |
| 6,855,345 | Jensen, et al. | Feb. 15, 2005 |
| 6,632,449 | Niehoff | Oct. 14, 2003 |
| 6,506,427 | Garti, et al. | Jan. 14, 2003 |
| 4,851,339 | Hills | Jul. 25, 1989 |
| 7,588,781 | Bortlik, et al. | Sep. 15, 2009 |

### CITED REFERENCES

1. Bailey L H. The Garden of the Gourds. New York: The Macmillian Company, 1937.

2. Baysal T., Ersus S., Starmans D. A J, Supercritical CO2 Extraction of˜-carotene and lycopene from tomato paste waste, J. Agric. Food Chem. 2000, 48, 5507-11.

3. Bendich, “Recent Advances in Clinical Research Involving Carotenoids,” Pure & Appi. Chem., vol. 66, No. 5, pp. 1017-1024 (1994).

4. Gartner C., Stahl W., Sies H., Lycopene is more bioavailable from tomato paste than from fresh tomatoes, Am J Clin Nutr 1997; 66:116-22, 1997.

5. Giovannucci et aI., “Intake of Carotenoids and Retinol in Relation to Risk of Prostate Cancer,” J. of Nat I. Cancer Inst., vol. 87, No. 23, pp. 1767-1776 (I 995). Heiser, Jr. The Gourd Book. Norman: University of Oklahoma Press, 1979.

6. Herklots GAC. Vegetables in South-East Asia. London: George Allen & Unwin LTD, 1972.

7. Khachik F. et ai. Effect of food preparation on qualitative and quantitative distribution of major carotenoid constituents of tomatoes and several green vegetables. J. Agric. Food Chem. 1992,40,390-8.

8. Mascia P, Kaiser S., Sies H., Lycopene as the Most Efficient Biological Carotenoid, Archives of Biochemistry and Biophysics, Vol. 274, No. 2, Nov I, pp. 532-8. 1989. Levy et aI., “Lycopene Is a More Potent Inhibitor of Human Cancer Cell Proliferation Than Either alpha-carotene, beta.-carotene,” Nutr. Cancer, vol. 24, pp. 257-266 (1995).

9. Le T. Vuong. Underutilized beta-carotene-rich crops of Vietnam. Food and Nutrition Bulletin, Vol. 21, No. 2, June 2000.

10. Le T. Vuong, Stephen R Dueker, Suzanne P. Murphy. Plasma beta-carotene and retinol concentrations of children increase after a 30-d supplementation with the fruit Momordica cochinchinensis (gac). Am J Clin Nutr 2002; 75:872-9.

11. Narisawa et ai., “Inhibitory effects of natural carotenoids, .alpha.-carotene, .beta.carotene, Iycopene and lutein, on colonic aberrant crypt foci formation in rats,” Cancer Letters, vol. 107, pp. 137-142 (1996).

12. Nguyen D V. Medicinal Plants of Vietnam, Cambodia and Laos. Westminster, Calif. Mekong Printing, 1998.

13. Perry L. M. Medicinal Plants of East and Southeast Asia, Attributed Properties and Uses. Cambridge: The MIT Press, 1980.

14. Sadler G., Davis J., Dezman D. Rapid extraction of lycopene and ˜-carotene from reconstituted tomato paste and pink grapefruit homogentes. J. Food Sci. 1990, 55, 1460-61.

15. Tan B. Analytical and preparative chromatography of tomato paste carotenoids. J. Food Sci. 1988,53(3),954-59.

16. Vuong, L. T. Xoi Gac, a rice preparation containing beta-carotene from Momordica cochinchinensis Spreng (gac), for the prevention of lycopene deficiency in northern Vietnam. Ph. D. Dissertation, University of California at Davis, 1998.

17. Ollanketo, M. et al., “Supercritical carbon dioxide extraction of lycopene in tomato skins,” Eur. Food Res. Technol. (2001) 212:561-565. cited by other.

18. Periago, M. J. et al., “Mixture Approach for Optimizing Lycopene Extraction from Tomato and Tomato Products,” J. Agric. Food Chem. (2004) 52:5796-5802.

19. Rozzi, N. L. et al., “Supercritical Fluid Extraction of Lycopene from Tomato Processing Byproducts,” J. Agric. Food Chem. (2002) 50:2638-2643.

20. Christensen et al., Res Commun Chem Pathol Pharmacol., vol. 50(1), pp. 147-150, 1985.

21. Li, 2002, Product development of sea buckthorn, Trends in new crops and new uses, pp. 393-398.

22. Vuong, A method of preserving and testing the acceptability of gac fruit oil, a good source of beta-carotene and essential fatty acid, 2003, Food Nutr Bull, 24, 372-3.

23. Vuong and King, “A method of preserving and testing the acceptability og gac fruit oil, a good cource of beta-carotene and essential fatty acids,” 2003, Food and Nutrition Bulletin, 24: 224-230.

24. Le T. Vuong, Stephen R. Dueker, and Suzanne P Murphy, Plasma .beta.-carotene and retinol concentrations of children increase after a 30-d supplementation with the fruit Momordica cochinchinensis (gac). American Society for Clinical Nutrition. 2002. 872-9.

25. Hiromitsu Aoki, Nguyen Thi Minh Kieu, Noriko Kuze, Kazue Tomisaka and Nguyen Van Chuyen, Carotenoid Pigments in GAC Fruit (Momordica cochinchinensis Spreng), Biosci. Biotechnol. Biochem., 2002, 2479-2482, 66(11).

What is claims is:

1. A process to obtain lycopene from the fruit Momordica cochinchinensis comprising the step of obtaining the ripen fruit of Momordica cochinchinensis, when the exocarp (peel) turns bright yellow and orange, further comprising the step of removing the seeds from the aril either manually, mechanically, by filtering, centrifugation, washing, with or without the use of water; further comprising the step of drying the wet components of the fruit by freeze drying, drum drying, tray drying, sun drying, or spray drying to reduce water to less than 10%; The drying process should be done in minimum exposure to light, air and in low temperature to avoid loss of lycopene and other carotenoids;

2. A process as claimed in claim 1, further comprising the step of pulverization, re-suspension, encapsulation, or emulsion using available equipments and published methods to improve stability and bio-availability of lycopene;

3. A process as claimed in claim 1 further comprising the step of extracting the oil from the aril using an expeller or oil press, and collecting the oil free membrane; further comprising the step of pulverizing the oil-free material to improve extraction, further comprising the step of extracting lycopene from the oil-free membrane and from the rind and pulp, by supercritical CO2 fluid extraction; water extraction, ethanol extraction, and/or centrifugation;

4. A process as claimed in claim 1, further comprising the step of combining the product with the oil fraction from the aril to get a higher concentration of lycopene, and to improve stability and bio-availability;

5. A process as claim in claim 3 further comprising the step of combining the lipophylic extract with whey protein, or soy powder, to increase solubility in aqueous solution;

6. A process as claimed in claim 1 by which the product may contain up to 45,000 ppm of lycopene, 220,000 ppm of total carotenoids, and 800 ppm of vitamin E.
